(19) United States
(12) Patent Application Publication
(10) Pub. No.: US 2005/0021155 A1
(43) Pub. Date: Jan. 27, 2005

(54) Device at a hand prosthesis

(76) Inventor: Stellan Brimalm, Vadstena (SE)

Correspondence Address: VENABLE, BAETJER, HOWARD AND CIVILETTI, LLP, P.O. BOX 34385, WASHINGTON, DC, 20043-9998, US

(21) Appl. No.: 10/488,009

(22) PCT Filed: Aug. 26, 2002
(86) PCT No.: PCT/SE02/01505

(30) Foreign Application Priority Data
Aug. 27, 2001 (SE) 0102831-5

(51) Int. Cl. A61F 2/48

(52) U.S. Cl. 623/64; 623/24

(57) ABSTRACT

Arrangement (1) for a hand prosthesis (2), which hand prosthesis (2) is adapted to replace a lost human hand on a human or artificial lower arm (3). The arrangement (1) is mounted between a wrist part (2a) and a lower arm (3). In addition the arrangement (1) has means for effecting tilting or turning of the hand part (2) relative to the lower arm (3).

### TECHNICAL FIELD

The present invention relates to an arrangement for producing a movement for a prosthesis hand relative to a prosthesis arm or a human arm. The arrangement is intended to replace a lost hand joint or wrist.

### BACKGROUND OF THE INVENTION AND RELEVANT PROBLEM

When a living hand or hand joint is lost or amputated it can be replaced by, respectively, a hand prosthesis or hand joint prosthesis. This means the loss of a satisfactorily operating body part having a realistic appearance, size, movement pattern and force assimilation capability.

The prosthesis that is to replace the lost body part should be simple to repair and should furthermore be inexpensive to produce.

One of the known hand prostheses comprises an unnaturally long wrist, which gives it an unrealistic appearance. The long length of the wrist prosthesis is due to the fact that in certain cases it includes a number of functions that a prosthesis hand has to perform, such as for example various movements of the parts of the prosthesis hand.

### OBJECT OF THE INVENTION AND SOLUTION OF THE PROBLEM

The object of the present invention is to solve some or other of the aforementioned problems.

This object is achieved with an arrangement according to claim 1 and a method according to claim 9.

In order for a hand prosthesis that is intended to replace a lost human hand and that comprises on the one hand a wrist part and on the other hand an arm part, to be able to control an arrangement between the wrist part and arm part, the arrangement having means for mutual tilting and turning between the wrist and arm part, the hand prosthesis receives a substantially natural movement pattern. Furthermore, by providing the arrangement with two substantially orthogonal axes the joint is enabled by means of two motors coupled to an arm cavity to execute on the one hand a tilting movement and on the other hand a turning movement, which movements may be executed together or individually.

### DESCRIPTION OF THE DRAWINGS

The invention will be described in more detail by means of an example of implementation and with reference to the accompanying drawings.

FIG. 1 is a diagrammatic perspective view of an arrangement according to the invention for a hand prosthesis with the hand part in an outstretched position.

FIG. 2 is a diagrammatic view according to FIG. 1, pivoted by 90° and showing the palm of the hand.

FIG. 3 is a diagrammatic view according to FIG. 1 seen from the side of the thumb with the hand part in an outstretched position.

FIG. 4a is a diagrammatic view according to FIG. 1 seen from the thumb side with the hand part in an upturned position. FIG. 4b is a diagrammatic view according to FIG. 4a but in the downwardly tilted position.

FIG. 5 is a diagrammatic view from above of an arrangement according to the invention.

FIG. 6 is a diagrammatic perspective view according to FIG. 5 in a position corresponding to that of a tilted hand part.

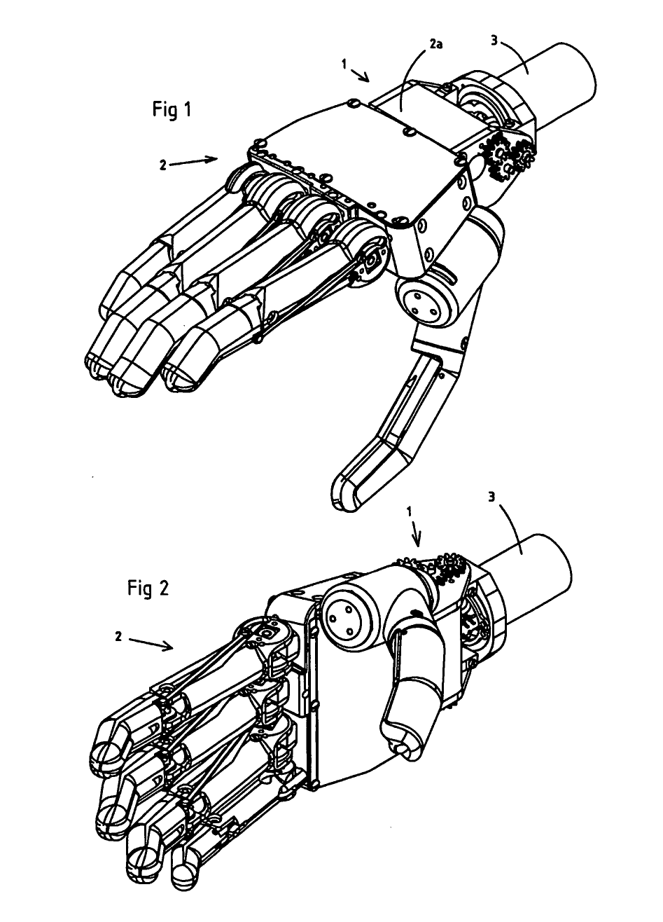

### DESCRIPTION OF THE EXAMPLE OF IMPLEMENTATION

An arrangement is generally denoted by the reference numeral 1 in the drawings. The arrangement 1 is arranged on a hand prosthesis 2 between a wrist part 2a and an artificial arm or arm part 2b, and comprises means 3 to permit mutual turning and/or tilting between the parts 2a, 2b, wherein the tilting or turning movements can be executed individually or together. These movements allow human hand movements to be simulated.

The word tilting is used hereinafter to distinguish it from turning, and involves pivoting a hand with respect to an arm or arm part in substantially the same way or direction of pivoting as the fingers of the hand are capable of turning or pivoting about their points of securement in the middle hand.

The two parts are substantially identical in shape and each comprises a U-shaped member, the U-shaped member having a base and two legs. The external part of the legs has a continuous opening in which is arranged a shaft 6a, 6b. The base of the U-shaped member is adapted to be arranged in the middle hand or pivotably arranged on the arm shaft 3b. The arm part 2b has a somewhat smaller distance between the legs of the U-shaped member so that it can be accommodated between the legs of the wrist part, the shafts 6a, 6b being arranged through a leg of respectively 2a, 2b. The shafts 6a, 6b are in this connection adapted to allow tilting of the wrist part 2a. On each one end of the two shafts 6a, 6b there is arranged outside the U-shaped member a transmission means 5 which connects the shaft 6a, 6b to motors 4a, 4b respectively, which latter are joined to the wrist part 2a. The motors 4a, 4b are preferably electrically driven and can be connected to a power source, for example a battery (not shown). On the two second ends of the shafts 6a, 6b, both of which are thus arranged between all four legs, there is a V-gear 7a, 7b, which V-gear 7a, 7b is joined to an arm cavity 8 that also includes V-teeth. Accordingly, when the V-gears 7a, 7b secured to the shafts 6a, 6b rotate in different directions around their common shafts this causes the said shafts 6a, 6b to rotate in the same direction around the arm cavity 8. This produces a turning of the wrist part relative to the arm cavity 8, for example in a position according to FIG. 1 and to a position 2, or vice versa. This turning movement can thus be effected by both motors 4a, 4b together. The magnitude of the turning movement is preferably ca. 150-180°.

In an alternative embodiment the transmission means 5 comprise toothed belts.

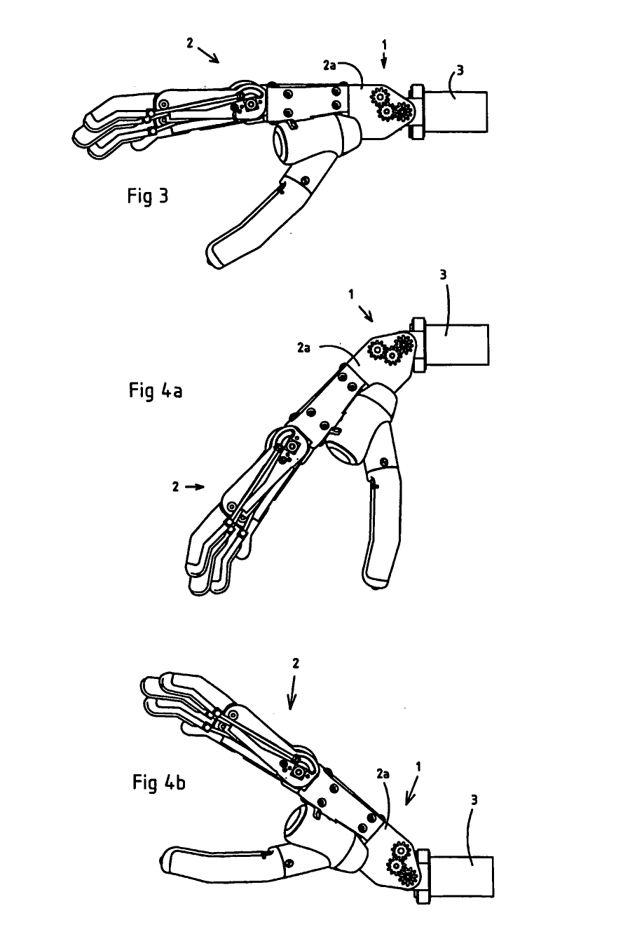

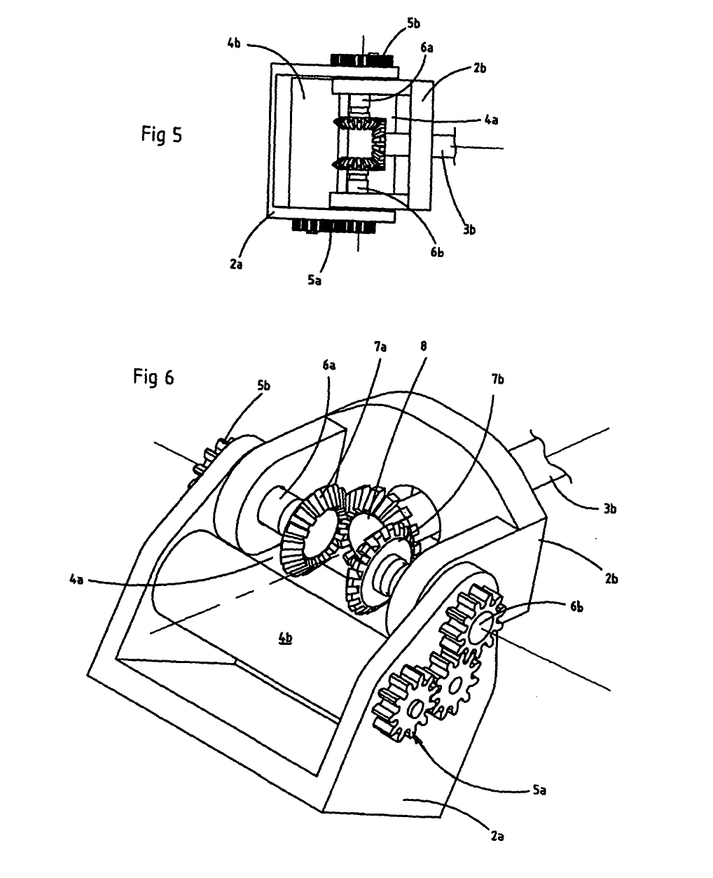

When the motors deliver power to drive the two shafts in the same direction of rotation with reference to their common longitudinal shafts, this means that the rotation of the shafts 6a, 6b relative to the arm part 2b is locked by the arm cavity 8 and the wrist part 2a can pivot relative to the shafts 6a, 6b, whereupon the wrist part 2a tilts and thereby tilts the whole hand prosthesis 2. Accordingly, the power supplied by both motors 4a, 4b can be used jointly to execute a tilting movement, as shown in FIGS. 3 and 4. The tilting movement may take place both upwardly and downwardly, as shown in FIGS. 4a and 4b, from an outstretched hand position, FIG. 3. The whole tilting movement preferably covers an angle of ca. 150°- 180°.

The motors 4a, 4b may also be driven so that they execute both turning and tilting movements in combination, i.e. so that the wrist is tilted and turned simultaneously. This means that, seen from the lower arm 3, the prosthesis hand should be able to execute not only an upward/downward tilting movement or pivoting in both directions around the arm shaft 3b, but should also be able to move obliquely upwards to the right, obliquely upwards to the left, obliquely downwards to the right and obliquely downwards to the left.

In order that the power delivered by the two motors is utilised simultaneously so as to effect tilting or turning, a maximum energy output can be ensured for the motors 4a, 4b and accordingly the space occupied by the motors can be effectively utilised.

In an alternative embodiment of the arrangement, the arm part and wrist part may for example change places, the wrist part then being the part that is accommodated within the arm shaft 3b .

The invention is not intended to be limited to the aforedescribed example, but can be varied within the scope of the claims.

SUMMARY LIST OF REFERENCE NUMERALS

1 arrangement
2 hand prosthesis
2a wrist part
2b arm part
3 lower arm
4 drive means
4a, 4b electric motors
5 transmission means
6a, 6b coaxial shafts
7a, 7b gear wheel
8 arm cavity

1. An arrangement for a hand prosthesis, which hand prosthesis is adapted to replace a lost human hand on a human or artificial lower arm and comprises on the one hand a wrist part and on the other hand an arm part, the arrangement being situated between the wrist part and the lower arm, wherein the arrangement has means to permit tilting and turning of the wrist part relative to the lower arm.

2. The arrangement according to claim 1, wherein the means comprise two substantially orthogonal shafts, around which the parts are capable of tilting or turning.

3. The arrangement according to claim 1, wherein the means permit simultaneous tilting and turning movements.

4. The arrangement according to claim 1, wherein the means comprise a drive unit.

5. The arrangement according to claim 4, wherein the drive unit comprises two electric motors.

6. The arrangement according to claim 4, wherein the means comprise a transmission adapted to convert the energy of the drive unit for tilting and/or turning movements of the hand prosthesis relative to the arm part.

7. The arrangement according to claim 4, wherein the motors are capable of executing jointly at least some of the turning and tilting movements.

8. The arrangement according to claim 4, wherein the drive device is connected to the wrist part and is adapted to drive via a transmission means two coaxial shafts accommodated on the wrist part, which shafts are in turn capable of actuating, via outer gears, an arm cavity connected to the arm shaft, around which the arm part is able to turn, and that the wrist part is capable of tilting around the two coaxial shafts.

9. A method of effecting a movement between a wrist part and an arm part, comprising:
arranging on the wrist part two controllable motors adapted to drive two coaxial shafts aligned therewith,
arranging on each shaft a gear in a gear-type engagement with an arm cavity connected to an arm shaft,
mounting the arm shaft in the arm part, and
controlling the motors such that the gears can rotate relative to the wrist part in the same direction in order to tilt the wrist part and/or so that the gears can rotate relative to the wrist part in different directions so as to turn the wrist part relative to the arm part.
